(19) United States
(12) Patent Application Publication
(10) Pub. No.: US 2012/0150451 A1
(43) Pub. Date: Jun. 14, 2012

(54) Optical Computation Fluid Analysis System and Method

(75) Inventors: Neal G. Skinner, Lewisville, TX (US); Michael T. Pelletier, Houston, TX (US)

(73) Assignee: Halliburton Energy Services, Inc., Houston, TX (US)

(21) Appl. No.: 13/324,185

(22) Filed: Dec. 13, 2011

Related U.S. Application Data
(60) Provisional application No. 61/422,475, filed on Dec. 13, 2010.

(51) Int. Cl. G06F 17/18; G01N 33/28; G06F 19/00; G01N 31/00

(52) U.S. Cl. 702/24; 702/179; 702/30; 702/25; 702/87

(57) ABSTRACT

Methods and apparatus for determining at least one property of fluids related to oilfield operations may include an optical calculation device for measuring light having interacted with the fluid (e.g., flowing fluids and flames). The flame may be fueled, at least in part, by the stream of fluid from the subsurface well. Methods may include directing interacted light that comprises light having passed through a fluid relating to an oilfield operation to an iris; performing a regression calculation on the interacted light with an optical calculation device responsive to the interacted light incident thereon to produce at least one output light signal; and determining at least one property of the fluid from the at least one output light signal.

### CROSS REFERENCE TO RELATED APPLICATION

The present application claims priority to U.S. Provisional Patent Application Ser. No. 61/422,475 filed on Dec. 13, 2010.

### BACKGROUND

The present invention relates to optical analysis systems and methods for analyzing fluids relating to an oilfield operation (for example, petroleum and drilling fluids), e.g., fluids flowing in a pipe or a well and fluids being extracted from a well or a reservoir.

Optical spectroscopy is an analytical technique that derives information about the system being evaluated by the interaction of that system with light in the ultraviolet (UV) to infrared (IR) range. The interaction changes the properties of the light, for example, the frequency (color), intensity, polarization, or direction (scattering or refraction).

Optical spectroscopy may be used to analyze samples in an oil field to characterize various samples. Hydrocarbon exploration and production may benefit from optical spectroscopy in the areas of flow assurances, real-time treatments of zones within a subterranean formation (e.g., the location and type of pill to be introduced into a wellbore), and pressure buildup that may lead to unwanted kicks.

Flow of fluids during oilfield operations can be hindered by, inter alia, wax deposition, asphaltene deposition, foaming, hydrate formation, corrosion, solid particles, reservoir erosion, and scale. Monitoring the production and buildup of these items that can lead to hindered flow may allow for an operator to take preventative steps before a plug, or the like, is formed. Plugging the flow often requires expensive and time-consuming remediation operations to get a wellbore back to a condition for continuing hydrocarbon exploration and production operations.

In a somewhat related area, determining petroleum quality while drilling may influence real-time decisions such as specialized treatments for individuals zones within a subterranean formation. Here, in addition to monitoring potential flow assurance issues, operators may use optical spectroscopy to monitor the quality of the hydrocarbons from the wellbore, e.g., water and H₂S concentrations. Real-time monitoring may allow for conducting plugging, diverting, or bridging operations to potentially undesirable zones as they are encountered.

In yet another application, optical spectroscopy may benefit operators working in underbalanced drilling operations. In underbalanced drilling operators drill through subterranean formations at pressures slightly less than the formation pressures, which can lead to hydrocarbons leaking from the formation into the drilling fluid. Moderate levels of hydrocarbons, e.g., levels low enough not to show a flame in a flare off, can yield a kick. In extreme cases, kicks can lead to wellbore blowouts and fires. Being able to identify the conditions that lead to a kick may allow operators to take preventative steps to minimize the strength of the kick, or more advantageously, avoid the kick.

While optical spectroscopy techniques have several potential applications in hydrocarbon exploration and production operations, the techniques are considered slow to permeate into the field. Optical spectroscopy can, in some instances, require precision optics, unique light sources, and precision alignment. These requirements often tend to be costly, slow, and cumbersome. Additionally, optical spectroscopy is generally not rugged enough for implementation in the field, which may demand frequent maintenance from persons with elevated levels of expertise. Further, the interpretation of complex spectroscopic results may, in some instances, necessitate persons with elevated levels of expertise.

A need is seen for a system and method for long-term monitoring of fluids during hydrocarbon exploration and production operations. Preferably, the system would be rugged and low cost with the ability to integrate into the various aspects of a complex petroleum field.

### SUMMARY OF THE INVENTION

The present invention relates to optical analysis systems and methods for analyzing fluids relating to an oilfield operation (for example, petroleum and drilling fluids), e.g., fluids flowing in a pipe or a well and fluids being extracted from a well or a reservoir.

In some embodiments, the present invention provides a method that comprises directing interacted light that comprises light having passed through a fluid relating to an oilfield operation to an iris; performing a regression calculation on the interacted light with an optical calculation device responsive to the interacted light incident thereon to produce at least one output light signal; and determining at least one property of the fluid from the at least one output light signal.

In other embodiments, the present invention provides a method that comprises directing interacted light that comprises light from a flame that comprises a fluid relating to an oilfield operation to an iris; performing a regression calculation on the interacted light with an optical calculation device responsive to the interacted light incident thereon to produce at least one output light signal; and determining at least one property from the at least one output light signal.

In still other embodiments, the present invention provides a system that comprises an interacted light comprising at least one selected from the group consisting of light having interacted with fluid relating to an oilfield operation, light emitted from a flame comprising fluid relating to an oilfield operation, and any combination thereof; an optical calculation device for performing a regression calculation on the interacted light, the device being responsive to the interacted light incident thereon to produce at least one output light signal; and a signal processing arrangement for determining at least one property of the fluid from the at least one output light signal.

The features and advantages of the present invention will be readily apparent to those skilled in the art upon a reading of the description of the preferred embodiments that follows.

### BRIEF DESCRIPTION OF THE DRAWINGS

The following figures are included to illustrate certain aspects of the present invention, and should not be viewed as exclusive embodiments. The subject matter disclosed is capable of considerable modifications, alterations, combinations, and equivalents in form and function, as will occur to those skilled in the art and having the benefit of this disclosure.

FIG. 1 is a schematic diagram of a nonlimiting example of a system for monitoring fluids relating to an oilfield operation employing in situ optical regression calculation devices according to an embodiment of the present disclosure.

FIG. 2 is a graph of absorbance vs. wavelength believed to be representative of crude broad band spectra.

FIG. 3 is a graph of heat of combustion in BTU/gm.

FIG. 4 is a side elevation sectional view of a nonlimiting example of an illustrative representative multivariant optical elements (MOE) filter construction.

FIGS. 5 and 6 are illustrative curves showing respective transmission and reflectance light intensity signature patterns of an illustrative substance property passed by a multilayered MOE filter.

FIG. 7 is a regression curve for the aromatics petroleum component property showing correlation of the measured and predicted values using partial least squares (PLS), wherein R²=0.97 shows the percentage variance captured.

FIG. 8 is a sectional elevation view of a nonlimiting example of a representative property monitoring system according to a further embodiment of the present disclosure and including a portion of the embodiment of FIG. 1.

FIG. 9 is a diagrammatic representation of a nonlimiting example of a system employing a plurality of MOE analysis devices for determining a property of a fluid relating to an oilfield operation sample employing both absorbance and fluorescence spectra and a plurality of MOE analyzing arrangements.

FIG. 10 is a diagrammatic representation of a nonlimiting example of a system employing a plurality of MOE analysis devices employed sequentially to measure the concentration of various chemical species burning in the flare.

FIG. 11 is a diagrammatic representation of a nonlimiting example of a system employing a plurality of MOE analysis employed simultaneously to measure the concentration of various chemical species burning in the flare.

### DETAILED DESCRIPTION

The present invention relates to optical analysis systems and methods for analyzing fluids relating to an oilfield operation (for example, petroleum and drilling fluids), e.g., fluids flowing in a pipe or a well and fluids being extracted from a well or a reservoir.

In some instances, there exists a need to evaluate fluids relating to an oilfield operation during drilling a wellbore, flowing fluids in a pipe to optimize decision-making during drilling, and monitoring flow assurance and other production characteristics of the fluid based on various parameters discussed above on-the-fly in real-time as quickly and as automatically as possible so that corrective or preventive action may be immediately taken. Conventional spectroscopic analysis techniques may not be very rugged and can be relatively costly, slow, and generally require sampling away from the wellsite or the pipeline.

The systems and methods of the present invention employing multivariate optical elements (“MOE”) may be useful for monitoring fluids in hydrocarbon exploration and production operations. An MOE is an optical computer that has advantageous ruggedness, low cost, and accuracy properties well-suited for application in analyzing and monitoring fluids relating to an oilfield operation, e.g., monitoring in a hostile environments. The systems and methods of the present invention may have particular application in environments of elevated temperatures and pressures or in remote and hostile settings including underground settings.

The systems and methods of the present invention employing MOE as described herein may further advantageously be used for real-time analysis and/or monitoring of fluids so as to provide data to operators to make on-the-fly changes to the compositions and methods involved in the associated wellbore operations, thereby increasing operation efficiency, reducing operational costs and downtime, and increasing worker safety by mitigating potentially detrimental results like kicks that can lead to blowouts and fires.

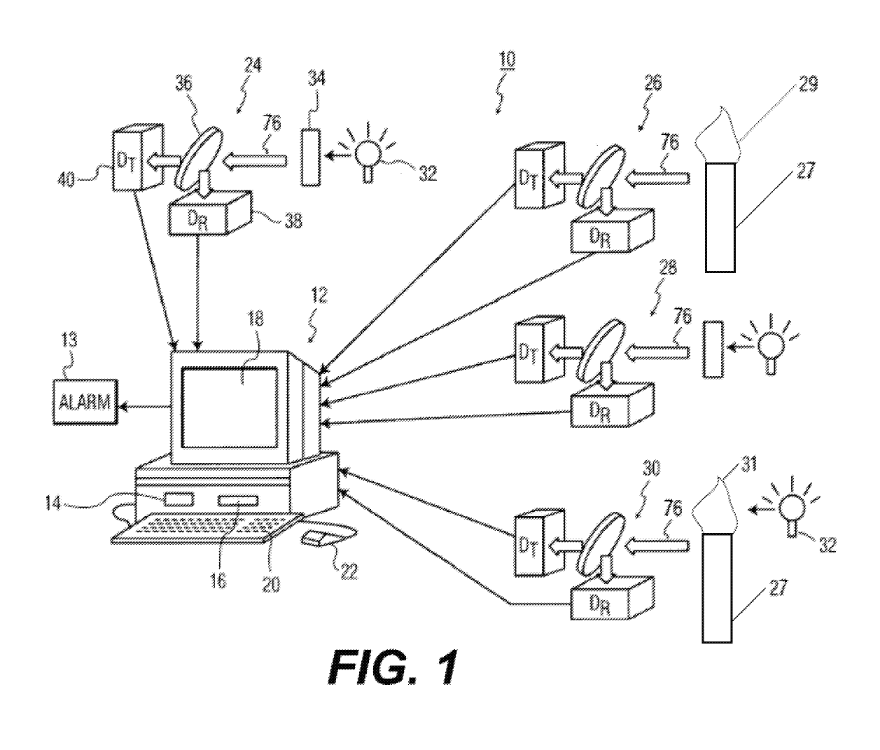

FIG. 1 provides a nonlimiting illustrative example of an MOE system of the present invention. System 10 may be used to determine a plurality of properties of fluids related to oilfield operations (e.g., drilling fluids, petroleum, hydrocarbon gases, and the like) while downhole in a drilling well, in a pipeline, or in a flare stack effluent whether they be flamed or not. When any of the determined properties are deemed to possibly affect the operation of the well (e.g., impact flow assurance or lead to a possible kick), system 10 may issue an alarm 13 as to that condition in real-time and identify the location of the condition. The term “property” means the chemical or physical characteristics or compositions of the fluid(s) relating to an oilfield operation. Example properties may include, but is not limited to, saturates, asphaltene, resins, aromatics, solid particulate composition and content (e.g., precipitates, dirt, mud, scale, and similar contaminants), H₂O ion-composition and content, fluid hydrocarbon composition and content, gas or volatile compositions and content (e.g., hydrocarbons with C₁-C₆ backbones, oxygen, CO₂, and H₂S), salinity, tracer concentrations, pH, and correlated PVT (pressure-volume-temperature) properties (e.g., GOR (gas-oil ratio), bubble point, density, a petroleum formation factor, and viscosity).

As used therein, the term “downhole” refers to being located in a well or a stream connected to a well or connected to any of one or more reservoirs whose fluids are subject to being pumped or otherwise moved to the surface at a well. In practice, numerous reservoirs may be interconnected by a web of streams all feeding a common wellhead.

As used therein, the term “pipeline” refers to a pipe employed to convey fluids relating to an oilfield operation, e.g., petroleum, from a field wellhead to a remote location. Pipes can be employed downhole and in pipelines. As used therein, the term “pipe” includes downhole pipes or pipeline pipes. Downhole pipes may be vertical or horizontal or at other spatial relationships.

As used therein, the term “residual” refers to that which is left over. In regression analysis, the residual is that part of the data that cannot be fitted by a regression.

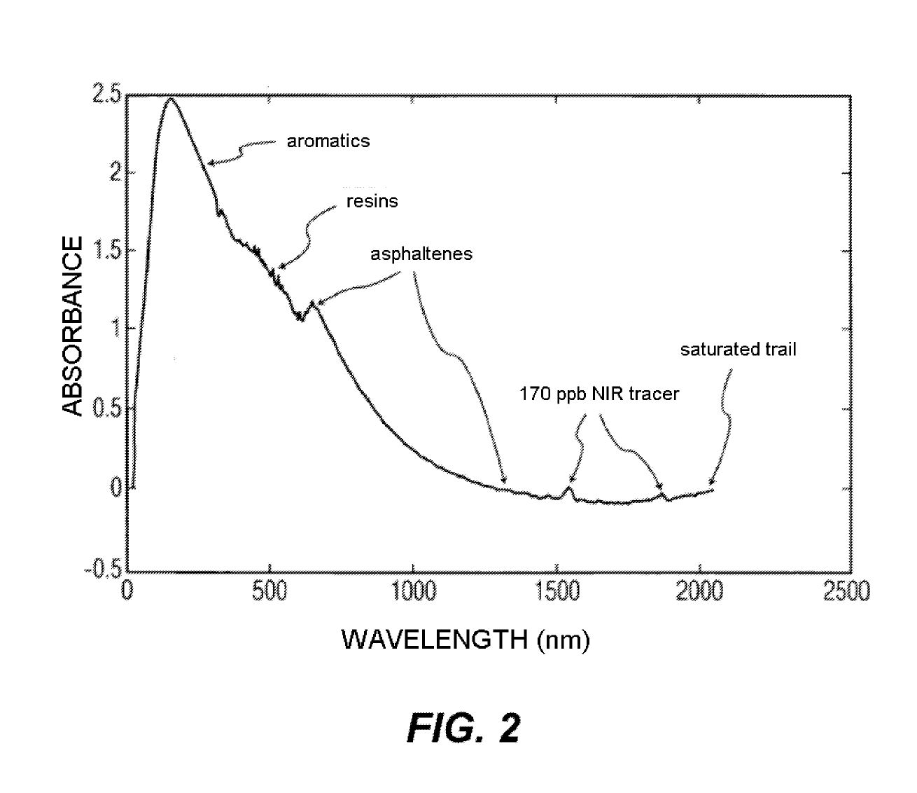

In FIG. 2, the broadband spectra of petroleum is shown to include aromatics, resins, asphaltenes, and saturates in which absorption is a function of wavelength. A 1270 ppb near infrared tracer (NIR) is shown at about 1500-1800 nm. The saturates are located at the high end of the spectrum.

Referring again to FIG. 1, system 10, unlike conventional systems, includes, in some embodiments, apparatuses located in a line of sight or otherwise in visual contact with flowing fluids related to oilfield operations. Components of system 10 may therefore in some embodiments be located downhole, on a pipeline, or remotely to determine in real-time the properties of the fluids related to oilfield operations that may be flowing in pipes underground, in the pipeline, or in a flare stack effluent whether they be flamed or not. As a result, the apparatuses of the system 10 may, in some embodiments, be subject to the extreme temperatures and pressures of the underground streams and/or the extreme temperatures of flares. Advantageously, systems of the present invention and components thereof employ rugged, reliable optical calculation devices as opposed to the costly spectroscopic instruments as used in conventional systems.

System 10 may, in some embodiments, include a programmed computer 12 including a microprocessor 14, memory 16 (which may include ROM and RAM for storing the analysis program), and an operating system program and determined data among other information as known in the art of spectral analysis, as understood by one of ordinary skill. The computer 12 may, in some embodiments, include a display 18, an input keypad 20, and a mouse 22. The computer 12 may, in some embodiments, be located at a central location remote from at least a substantial portion of the system 10 comprising optical sensing and optical calculating analyzing devices 24, 26, 28, and 30. While four devices are illustrated in FIG. 1, the number of devices is by way of illustration and more or fewer devices may be used in practice. One skilled in the art should understand the relative necessary number of devices and acceptable relational configuration of the devices to obtain the desired results.

Each device 24, 26, 28, and 30 may detect and analyze light having passed through fluids related to oilfield operations of interest, referred to herein as “interacted light.” In some embodiments, interacted light may include light having interacted with fluids relating to an oilfield operation, light emitted from a flame comprising fluids relating to an oilfield operation, and/or any combination thereof.

The system 10 may, in some embodiments, have as many devices as properties that one desires to measure for the fluids related to oilfield operations, each device corresponding to a different property. The devices may, in some embodiments, all be located at or adjacent to a common fluid stream located in the field or pipeline to provide an overall picture of the condition of the fluid at that location. It should be noted that devices may be compacted into individual system components, i.e., the term “devices” does not imply a plurality of individual devices.

If the desired property determination is of a type and magnitude determined to affect the operation of the well, the computer 12 may, in some embodiments, be programmed to generate an alarm signal and to activate an alarm 13. The alarm 13 may include audio, visual, or other indicators such as sirens, a light, either blinking or steady state, a display, an electronic signal to another computer, an electronic signal to a data acquisition system, an electronic signal to a supervisory control and data acquisition (SCADA) system, an electronic signal to a control system, an electronic signal to a monitoring system, or any combination thereof. Indicators for the alarm 13 may be located in the field at any number of different sites (e.g., at or near the device and/or remotely) for immediately alerting personnel to a detected immediate problem flow assurance condition. The computer 12 may determine an unacceptable property to affect the operation of the well detected by the associated device(s). The alarm 13 may alert personnel of the existence of a detected problem. The computer 12 may be programmed to provide information relating to the location and the nature of the problem based on the location of the devices and associated apparatuses in the system 10. In some embodiments, the computer may be programmed to correct any detected problem. For example, the computer may be programmed to shut down a well if it is emitting an unacceptable level of H₂S, or choke back to prevent or mitigate the strength of a kick if an unacceptably high rate of hydrocarbon production is detected.

In some embodiments, the devices 24, 26, 28 and 30 may be rugged, so as to be capable of withstanding the temperatures and pressures in situ in the desired location relative to fluids relating to an oilfield operation, e.g., capable of withstanding temperatures and pressures at the pipes or the temperatures at the flares, and thus may be located for long-term permanent use or for short-term temporary use, depending on the particular application. These devices may be a marked improvement over conventional analyzing systems, which may not be very rugged, may not be automatic and may not be acceptable for long-term use, but rather may be limited to short-term use. In some embodiments, devices (or the environment around devices) may be cooled to mitigate deleterious effects from high temperatures and pressures. In some embodiments, device placement may be adjusted and/or designed so as to mitigate and/or minimize deleterious effects from high temperatures and pressures, e.g., mounting devices at the top of a flare stack at some distance away from the flare itself.

The number of devices as illustrated in FIG. 1 is arbitrary and is not intended to convey any significance. The number of devices in a system depends also on the number of locations, e.g., pipes and/or flares, as well as the number of properties being monitored and/or analyzed at each location. Further, the number of devices of a system may not necessarily be limited to measuring the properties of fluids related to oilfield operations in a single location. By way of nonlimiting example, devices may be mounted to monitor the effluent from a flare stack at different points in the effluent. In some embodiments, the computer 12 may be programmed to monitor a large number of devices associated with and located in respect of a number of different fluid streams, pipes, or flares. Thus, the properties of fluids related to oilfield operations flowing independently in different pipes or flares may be monitored simultaneously by one or more computers 12, which may also be programmed to correlate a number of different properties as being related to a flow assurance problem in one reservoir system.

The computer 12 may, in some embodiments, be coupled to an alarm 13, which may provide an audible alarm, a visual alarm, or both. By way of nonlimiting example, a map layout of the petroleum field or pipeline distribution network may have the various branch fluid streams identified by code or some form of labeling. The optical analysis devices may each be associated with a code identifying the particular fluid stream, pipe, or flare stack it monitors. The computer 12 may, in some embodiments, analyze the optical analysis inputs for non-acceptable property situations, and may output the nature of the property and its location. With this information, immediate corrective action may be taken.

In one embodiment of the present invention, one computer 12 may be used to monitor pipes in interconnected fluid streams and reservoirs to provide an overall picture of the fluids flowing to a common wellhead, for example. In this way, problems associated with one stream may be readily associated with corresponding fluid streams feeding that one fluid stream. The devices may monitor similar properties of the different fluid streams to determine the source of the problem property, e.g., the fluid stream(s) and/or reservoirs(s) exhibiting the same potential problem. Thus, in some embodiments, the computer may be programmed to display and indicate a possible source of a problem by analyzing similar properties from common interconnected fluid streams and/or reservoirs. Advantageously, this may readily simplify problems of which streams to monitor and log as compared to costly and complex conventional spectroscopic systems.

Each of the devices 24, 26, 28, and 30 may have similar elements so that a description of one device 24 is representative. Each device may be arranged to determine at least one property of a fluid related to oilfield operations. The properties determined include any of those depicted above, but preferably include at least the more important constituents where importance is field dependent. Other properties, of course, may also be sensed and detected by still other devices (not shown in FIG. 1).

It should be understood that a given pipe or flare stack may be associated with as many devices as deemed appropriate for a given fluid stream, location, and reservoir. It is known, for example, that different reservoirs may exhibit fluids and solids with different detrimental properties such as chemical potential for scale, watering, hydrates, etc. In some embodiments, once the properties are determined for a given reservoir or fluids relating to oilfield operations, it may not be necessary to monitor for all properties depending upon the findings relative to that reservoir, fluid stream, pipeline, and so on. Such properties may, in some embodiments, be monitored on a scheduled basis, for example, using a pig (i.e., a device including but not limited to a robot designed to go downhole or in a pipeline and perform work) carrying one or more devices such as device 24 or may be fixed in place relative to each fluid stream.

The devices 24, 26, 28, and 30 may be relatively low cost and rugged, and thus, capable of implementation in many more locations and fluid streams than otherwise possible with conventional systems.

Representative device 24 for use in conjunction with systems and methods of the present invention may, in some embodiments, have a light source 32, a transparent sample cell (or series of windows) containing the fluid 34 being monitored that may be flowing as a fluid stream or static, an MOE 36 that may be an optical regression calculation device, a detector 38 for detecting light reflected from MOE 36, and a detector 40 for detecting the light transmitted by MOE 36. The MOE 36 may, in some embodiments, be a unique optical calculation device that comprises multiple layers.

Nonlimiting examples of light sources may include light emitting diodes, broad wavelength sources, narrow wavelength sources, single wavelength sources, white lights, colored lights, infrared lights, near infrared lights, ultraviolet lights, and the like. It should be noted that the term “light source” does not imply a single source of light or single wavelength of light. In some embodiments, a light source may be a plurality of light emitting sources.

In some embodiments, representative device 26 may have a light source in the form of a flame 29 to detect the effluent from a flare stack being monitored that may be flowing from a flare stack 27 with detectors analogous to those described with respect to representative device 24. In other words, the flame 29 burning at a top of flare stack 27 provides the light source and the sample.

In some embodiments, the flame 29 may be fueled, at least in part, by fluid from a well operation. For example, in some well operations, such as underbalanced drilling, fluid produced from the well may include mud, oil, gas, and water. In some embodiments, a portion of the mud may be separated from the remainder of the fluid and recycled, then oil may be separated out and stored. Likewise, gas and water may be separated, with the gas directed to the flare stack 27 and burned as flame 29, while the water may be stored. While the flame 29 may primarily use gas as fuel, it may also use any other fuel, whether provided by the well operation or otherwise. Determination of the chemical makeup of the flame 29 may provide insight as to the chemical and physical properties of the flared material as well as the formation. When this determination is made quickly, real-time data may allow for building an accurate geological model, providing safety and enhanced well operations.

In some embodiments, representative device 30 may have a light source 32 on one side of the effluent 31 from a flare stack 27 being monitored that may be flowing from a flare stack 27 with detectors analogous to those described with respect to representative device 24. In other words, the effluent 31 from a flare stack 27 may not need to be burning to analyze the properties of the effluent 31. In some embodiments, the flare stack may have a light source and device and be capable of being flamed, i.e., the device may be capable of detecting properties if the flare stack effluent is burning or not. One skilled in the art should recognize that different MOEs may be necessary to detect the similar properties if the flare stack effluent is burning as opposed to not.

In some embodiments, detection and analysis of properties of a fluid relating to oilfield operations may be conducted at some distance from the sample. Thus, the apparatus described herein may not require a physical connection to equipment at the site. Such apparatus may, in some embodiments, be portable, requiring no special parts, and providing no impact on existing equipment. In some instances, the apparatus described herein may be mounted on or present in a truck configured to readily move from one work site to another.

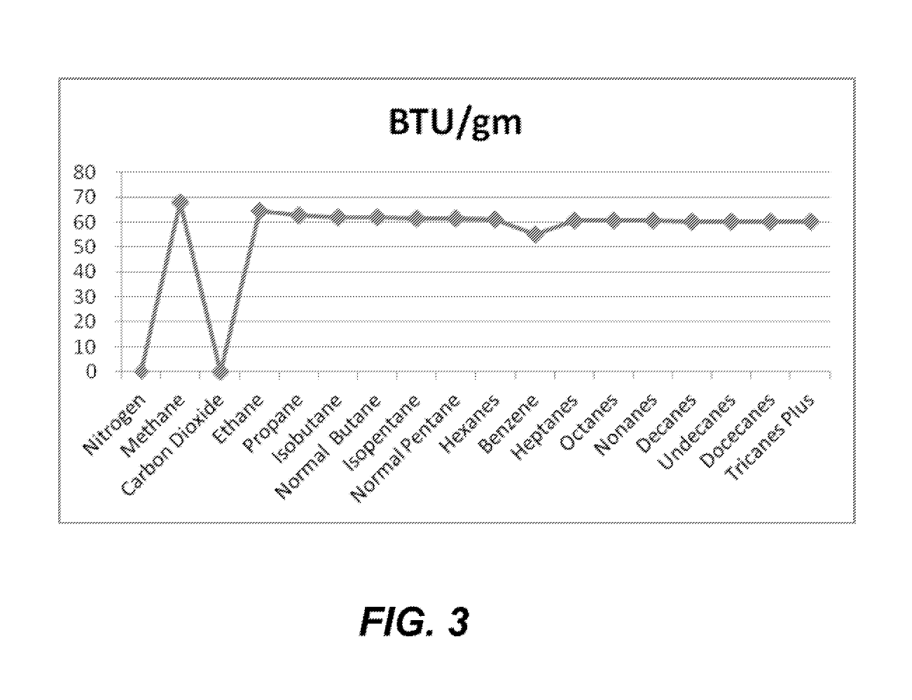

By way of nonlimiting example, the volume of the flame 29 may be estimated. In some instances, two or more cameras directed toward the fluid (e.g., flame 29) may provide a reasonable estimate of the volume. The temperature of the flame 29 may also be available (e.g., via an infrared thermometer on the flame), allowing the total amount of heat produced by the flame to be readily calculated. Based on the heat produced, the amount of fuel being burned may be calculated on a weight basis. This calculation may be used in conjunction with the determination of chemical makeup to estimate a hydrocarbon flow rate. For hydrocarbons, the specific heat of combustion (on a weight basis) is nearly constant, as indicated in FIG. 3.

One problem associated with conventional well testing is the separation of hydrocarbon mist from the gas stream at the separators. The entrained liquid is frequently under-counted in the liquid tally from the separator and may not be properly metered by the orifice plate type meters in the gas metering leg of the separator. Because the gas and entrained liquid are typically sent to the flare stack 27, the total heat of combustion approach outlined above may account for this under-counted material.

An analysis of CO₂ may be carried out to correct the total gas flow. The CO₂ in the gas stream from the reservoir may not contribute as a fuel, but may reduce the gas flame temperature, as some heat will be diverted to heating the original CO₂ content to the (lower) flame temperature.

The methods described herein may, in some embodiments, allow for a better estimate of how much mist is passing through the flare stack 27. These estimates may be compared with estimates of the level of liquid phase (i.e., mist) expected in the flare stack 27 to determine whether separation parameters should be modified. Thus, adjustments may be made in the separators or other equipment upstream of the flare stack 27.

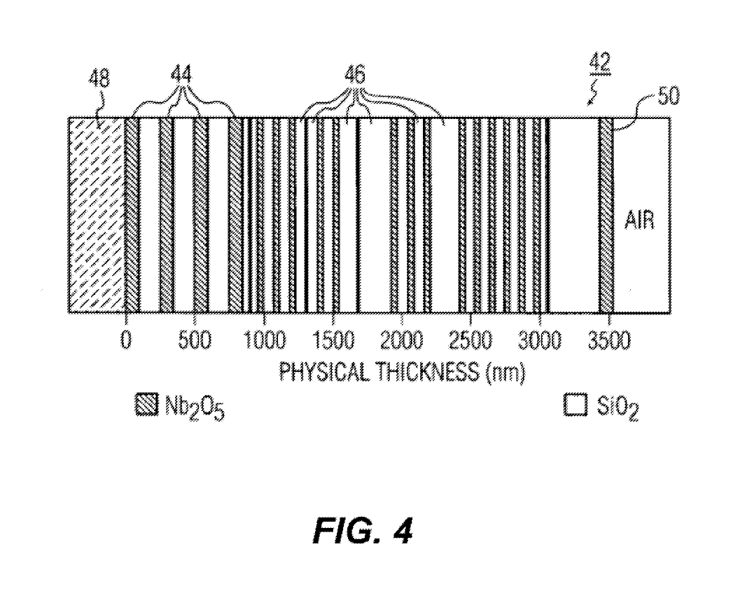

As illustrated in FIG. 4, for example, representative optical regression calculating device MOE 42 for use in conjunction with the present invention may have alternating layers 44 and 46 of Nb₂O₅ and SiO₂ (quartz). The materials making up the alternating layers are but one example of a pair of suitable materials which may be used in the construction of the MOE filter. Other material may be stacked to produce the desired reflection and transmission spectra, however, the thickness of the individual layers will be material dependent. The main requirement for the materials making up the stack is that individually, they are transparent to the desired range of wavelengths and that the stacked materials have different refractive indices. Additionally, three or more different materials may be included in the stack, however this complicates the manufacturing and analysis of these devices. The layers may be deposited on any optical substrate 48, such as, for example, a transparent substrate such as the type referred to in the art as BK-7, silica, quartz, or sapphire.

The spectrum of interest of a given property may include any number of different wavelengths. It should be understood that the MOE 42 of FIG. 4 is provided for purposes of illustration only. The number of layers and their relative thicknesses of FIG. 4 thus bear no correlation to any fluid property to which the present disclosure is directed and are not to scale. The thickness of the layers may be in the order of microns each as shown.

The multiple layers may have different refractive indices. By properly selecting the materials of the layers and their spacing, the optical calculation device may be constructed so as to selectively pass predetermined fractions of light at different wavelengths. Each wavelength may be given a predetermined weighting or loading factor. The thicknesses and spacing of the layers may be determined using a variety of approximation methods from the spectroscopic of the property of interest. These methods may include inverse Fourier transform (“IFT”) of the optical transmission spectrum and structuring the optical calculation device as the physical representation of the IFT. The approximations convert the IFT into a structure based on known materials with constant refractive indices. The structures of such optical calculation devices is provided at Applied Optics, Vol. 35, pp. 5484-5492 (1996) and Vol. 129, pp. 2876-2893, incorporated by reference herein.

The weightings that the MOE 42 layers apply at each wavelength may be set to the regression weightings described with respect to a known equation, data, or spectral signature. The optical calculation device MOE 42 may perform the dot product of the input light beam into the optical calculation device and a desired loaded regression vector represented by each layer for each wavelength. The MOE 42 output light intensity may be directly related to and proportional to the desired fluid property. The output intensity may represent the summation of all of the dot products of the passed wavelengths and corresponding vectors.

By way of nonlimiting example, if the property being measured is resin presence in petroleum and the regression vectors correspond to the resin, the intensity of the light output of the MOE would be proportional to the amount of resin in the sample through which the light beam input to the optical calculation device would have passed or would have been reflected from or otherwise interacted with. The ensemble of layers would correspond to the signature of resin. These wavelengths would be weighted proportionately by the construct of the corresponding optical calculation device layers. The resulting layers together would produce an optical calculation device MOE 42 output light intensity from the input beam. The output light intensity would represent a summation of all of the wavelengths dot products and the loaded vectors of that property, e.g., resin. The output optical calculation device intensity value would be proportional to the amount of resin in the petroleum being examined. In this way, an MOE optical calculation device may be produced for each property to be determined in fluids relating to oilfield operations.

While FIG. 4 illustrates one type of MOE, various other optical computing devices may be considered an MOE in accordance with the present disclosure. For example, other suitable MOE devices for use in conjunction with the present invention may, in some embodiments, be programmable with mirrored arrays, or may have variable programming to detect various chemical makeups.

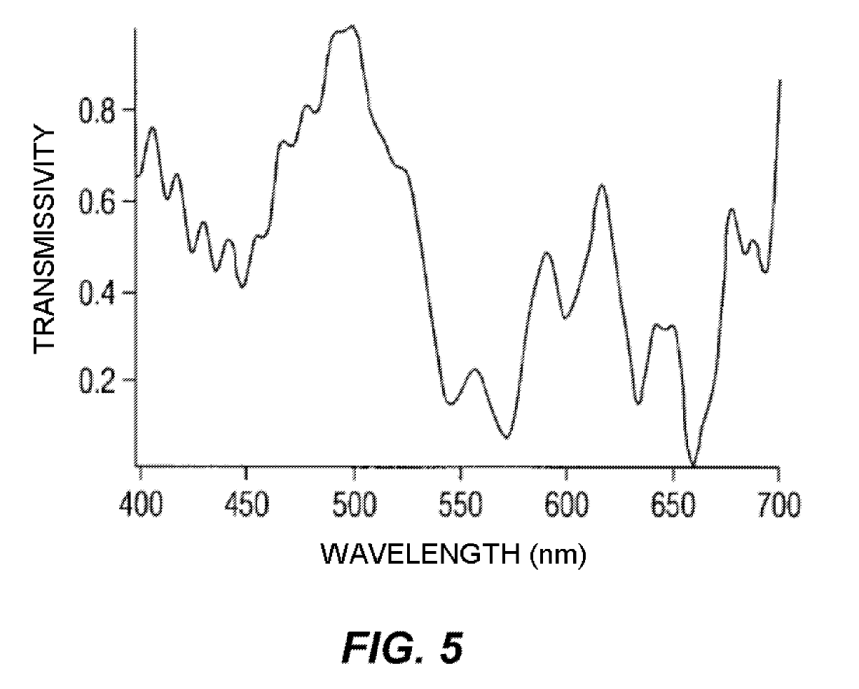

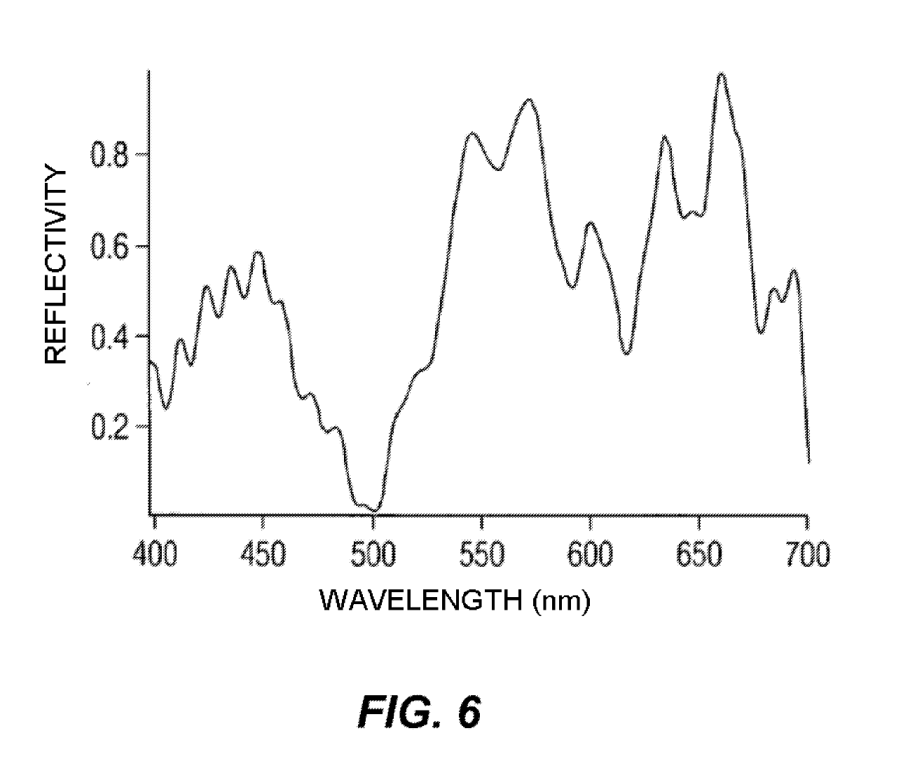

Such MOE optical calculation devices represent pattern recognition devices that produce characteristic output patterns representing a signature of the spectral elements that define the property of interest. The intensity of the light output is a measure of the proportional amount of the property in the test media being evaluated. For example, an MOE transmission output waveform might appear as in FIGS. 5 and 6, which do not represent any specific fluid property, but are shown for purposes of illustration only. The waveform of FIG. 5 forms a pattern comprising different wavelengths of a spectrum that is unique to a property. This waveform may be the light that impinges upon detector 40 (shown in FIG. 1), for example, or the corresponding detectors of devices 26, 28, and 30 of the system 10. Each of these detectors, such as detectors 38 and 40 associated with MOE 36, may transmit its output (e.g., an electrical signal) representing the magnitude of the intensity of the signal of FIG. 5 that is incident on the detector. This signal may thus be a summation of all of the intensities of the different wavelengths incident on the detector. The various weighting factors assigned to each layer produce a composite signature waveform for that property.

The reflected light from the MOE 36 (shown in FIG. 1) may produce a negative of the transmitted signal of FIG. 5 for no sample or optical absorbance. This reflected signal may be represented by the waveform of FIG. 6 and may not be a regression calculation. The computer 12 (shown in FIG. 1) may subtract the reflected signal from the transmitted signal of FIG. 5. The difference may represent the magnitude of the net light intensity output from the MOE and the property in the fluid being examined. This subtraction provides correlation that may be independent of fluctuations of the intensity of the original light due to power fluctuations, use of different light bulbs of the same type as used in the original apparatus, or variations in intensity or direction of the flame 29 emitted from the flare stack 27. That is, if the transmitted light intensity varies due to fluctuations, the system could interpret this as a change in property. By subtracting the negative reflections, the result is an absolute value independent of such fluctuations and thus provides needed correlation to the desired property being determined. Either the raw detector outputs may be sent to a computer, or the signals may be subtracted with an analog circuit and magnified with an operational amplifier converted to voltage and sent to the computer as a proportional signal, for example.

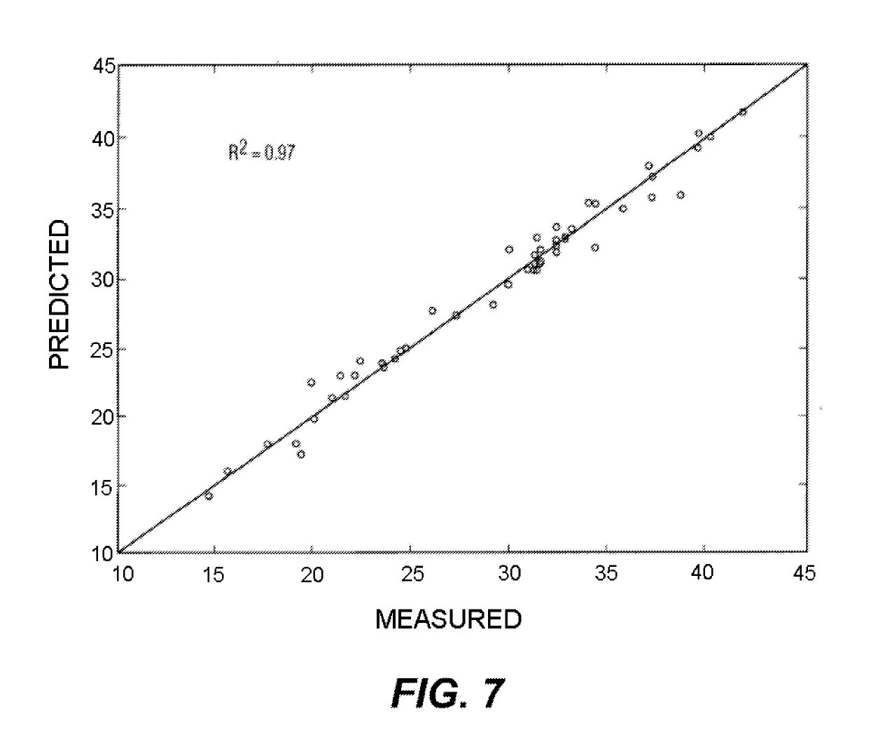

There is good correlation between the predicted properties, such as aromatics, for example, in FIG. 7, and the measured amount of property. Thus some embodiments of the present invention for determining at least one property of fluids related to oilfield operations may comprise causing the fluid to produce a flame (e.g., adding fuel, lighting the fluid, entraining the fluid in a flame, and the like), which is passed through a telescope to create interacted light; performing a regression calculation on the interacted light with an optical calculation device responsive to the interacted light incident thereon to produce at least one output light signal manifesting the calculation and the corresponding at least one property; and determining the at least one property from the at least one output light signal.

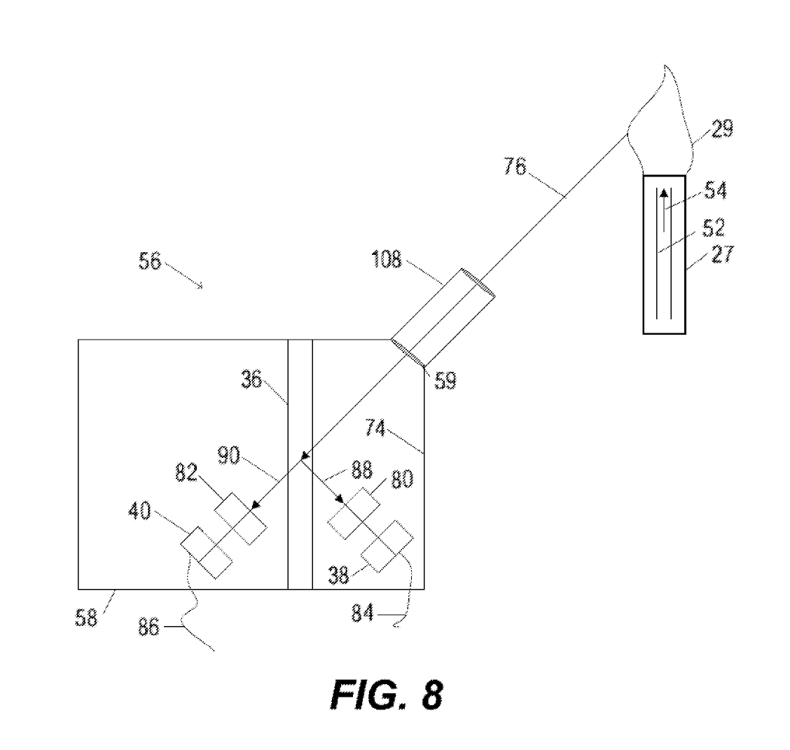

Referring now to FIG. 8, which provides a nonlimiting example of a system of the present invention, representative flare stack 27 may, in some embodiments, be connected to a fluid stream, e.g., a downhole pipe. For example, the downhole pipe may be a vertical pipe or horizontal pipe and may be part of the wellhead or interconnecting stream pipes that interconnect various reservoirs of petroleum in a field. Fluid 52 and/or associated water gas (or in some embodiments, other fluids relating to oilfield operations) may flow in the pipe in direction 54. In some embodiments, aligned with the flare stack 27 may be a portable or fixed optical MOE fluid property detecting apparatus 56. In some embodiments, apparatus 56 may correspond to any one or more of the devices 24, 26, 28, and 30 of FIG. 1, and may be utilized in a system such as system 10 shown in FIG. 1 for determining the amount of a property of the fluid flowing from the flare stack 27. In some embodiments, the system of the present invention corresponding to system 10 may utilize the apparatus 56 to determine the amount of the property in real-time and report that amount instantaneously as it occurs in the flowing stream of fluid 52 and simultaneously with other apparatuses (not shown) corresponding to apparatus 56 aligned with flare stack 27 adjacent to or at a given location near the flare stack 27 and still other pipes corresponding to numerous steams in the field being monitored.

In some embodiments, the apparatus 56 may have a housing 58 having any of a number of shapes, such as cylindrical or rectangular. The MOE 36 may be bonded to the housing 58, for example, or attached in other ways as known in this art. A light source (e.g., in the form of the flame 29) may be aligned using a telescope 108 or other mechanism so as to pass the interacted light 76 through an iris 59 allowing the amount of light entering the housing cavity 74 to be controlled. As used herein, the term “iris” refers generally to devices for limiting the total intensity of the light and encompasses slits, cross-polarized filters, neutral density filters, holographic filters, specked beam splitters, and apertures, which may in some embodiments be adjustable or fixed. While the telescope 108 is illustrated as connected to the housing 58, other configurations may include a telescope detached from or otherwise situated near the housing 58. Likewise, even though the iris 59 is illustrated as a component of the telescope 108, other configurations may include an iris detached from the telescope 108. Further, a window or other covering may be provided between the iris 59 and the environment, to isolate the contents of the housing 58 from external conditions. Upon entering the housing 58, a portion of the interacted light 76 emitted from the flame 29 or other light source may be transmitted to detector 40 and another portion of the interacted light 76 may be reflected to detector 38.

Located in the cavity 74 of the housing 58 may be one or more MOE optical calculation devices 80 and 82 and detectors 38 and 40 responsive to the output of the MOE optical calculation devices 80 and 82 for generating corresponding electrical intensity output signals whose difference corresponds to a property of the fluid to be determined. Conductors 84, 86 may receive the detector output signals. Wires such as conductors 84, 86 may be connected to a computer such as computer 12 (shown in FIG. 1) located at a remote station for determining the property of the fluid manifested by the signal on conductors 84 and 86. In the alternative to hard wiring the apparatus 56 components, a battery, a local generator, or telemetry could also be used to power the apparatus components.

In some embodiments, in operation of the apparatus 56 (shown in FIG. 8, a nonlimiting example of a system of the present invention), the interacted light 76 from the flame 29 passes into the apparatus via the iris 59. The behavior of the interacted light 76 will be dependent on the chemical makeup of the fluid or other material passing from the flare stack 27 and providing fuel for the flame 29. As the light hits the MOE 36, a portion of the interacted light 76 is transmitted through, and a portion of the interacted light 76 is reflected from the MOE 36. The particular configuration of MOE 36 may change depending upon the fluid type and composition, gas phase, water phase, the component being analyzed and so on. As a result, the interacted light 76 from the flame 29 is split into reflected light 88 and transmitted light 90. The reflected light 88 and the transmitted light 90 pass through respective MOE optical calculation devices 80 and 82 to respective detectors 38 and 40.

In some embodiments, a separate apparatus 56 may be provided for each property to be determined from the flowing fluid. In some embodiments of the present invention additional apparatus are provided in the same housing, with various components therein sharing the interacted light 76 at the same time. In other embodiments, additional apparatus may be provided in the same housing, with various components therein being exposed to the interacted light 76 in turn. In some embodiments, fiber optics (not shown) may be used in the apparatus 56 as part of the light paths.

However, it should be understood that the distribution of light associated with the various light paths between the light source (e.g., flame 29 as shown in FIG. 8 or light source 32 as shown in FIG. 1), and the apparatus 56 is critical to the construction of the MOE 36. That is, different housings, and light sources associated therewith all have unique light paths that may affect the light distribution. These light paths and distributions need to be taken into consideration during the MOE construction. This construction is based on a representative spectrum for the fluid of interest. The intensities and distribution of the various wavelengths may vary from apparatus to apparatus, and thus, such light paths and distributions need to be taken into consideration in the design and construction of the MOE associated with a given apparatus.

In some embodiments, this problem may be resolved by using the housing 58 and flame 29 that will be utilized for the optics associated with a given MOE for use in generating the spectrum that is to be provided by a conventional spectrometer. That generated spectral data is then utilized to construct the MOE that is to be utilized with the associated housing and light source. Thus, it may be assured that the light paths and distributions from the installed housing and light source are identical to those used to create the MOE, and thus, there will be no errors or problems in utilizing the MOE with such components. Thus if such components ever need replacement, a new MOE may be constructed unique to those replacement components or otherwise compensated for changes in light distributions. Otherwise an error in property determination may be possible if other components are utilized other than those used to create the MOE. Thus, any components utilized in or that may affect the optical path lengths or wavelength distributions from light source to MOE need to be utilized to determine the spectral aspects of the property of interest used to construct the corresponding MOE.

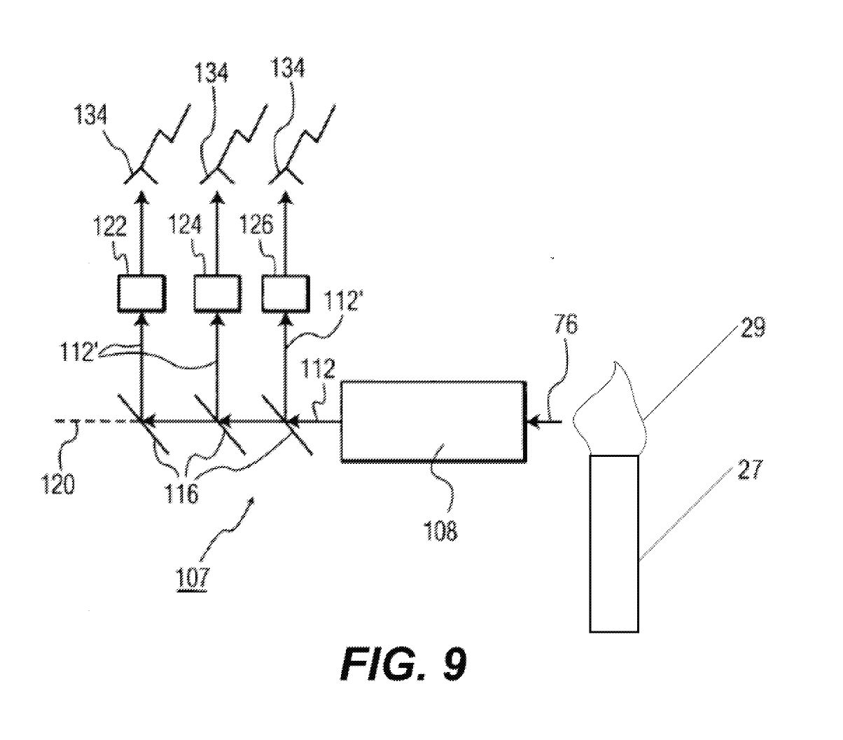

Referring now to FIG. 9, an alternative embodiment is illustrated utilizing a nonlimiting example of a system 107 of the present invention. System 107 may include a telescope 108 or other apparatus for concentrating, limiting, controlling field of view, excluding background, polarizing, filtering, enhancing the contrast of, or otherwise conditioning, the interacted light 76, e.g., to include only the interacted light 76 from the flame 29, or a portion thereof. The flame 29 may provide light to the telescope 108, resulting in a beam 112 of focused light. Beam 112 may be reflected from mirrors 116 and redirected at about 90°. The mirrors 116 may be partially transmissive forming partially reflected beams 112′. While three beams 112′ are shown, in practice any number of beams may be utilized as represented by the dashed line 120. The reflected and transmitted beams may not be frequency biased.

The reflected beams 112′ can be directed to one or more corresponding MOEs 122, 124 and 126. The output signals of the MOEs can be detected by detectors 134 whose outputs can be analyzed by a computer (not shown). By way of example, four different spectrums may be analyzed by the absorbance MOEs 122-126 and so on. The system 107 may thus obtain four different measurements on four different properties, asphaltene, resins etc. at the same time. The outputs of the detectors 134 associated with beam 112 results in a property that is proportional to the sum of two MOE detected signals, one from each spectroscopic absorbent component which represent one property. Thus, four different properties are analyzed with the beam 112.

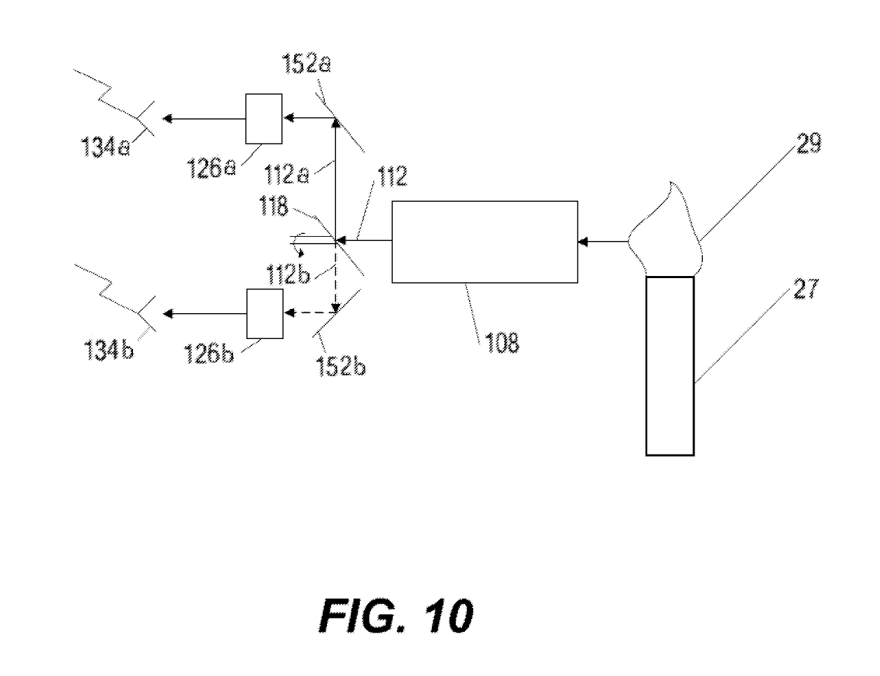

Referring now to FIG. 10, which provides a nonlimiting example of a system of the present invention, a plurality of MOEs may be selected, in turn, through the use of a wheel on which the MOE devices sit, or through the use of a rotating mirror 118. Thus, beam 112 may leave telescope 108 and interact with the rotating mirror 118. Depending on the orientation of the rotating mirror 118, beam 112 may be diverted in a direction 112a, 112b, or any of a number of other directions (not shown). Beam (e.g., 112a, 112b) may then pass directly to one or more MOEs and on to corresponding detectors, or, as illustrated, it may interact with one or more additional mirrors 152a, 152b, which may be fixed, before passing to MOEs 126a, 126b and detector(s) 134a, 134b. In this manner, rotation of the rotating mirror 118 may allow for any number of MOEs to be used in series.

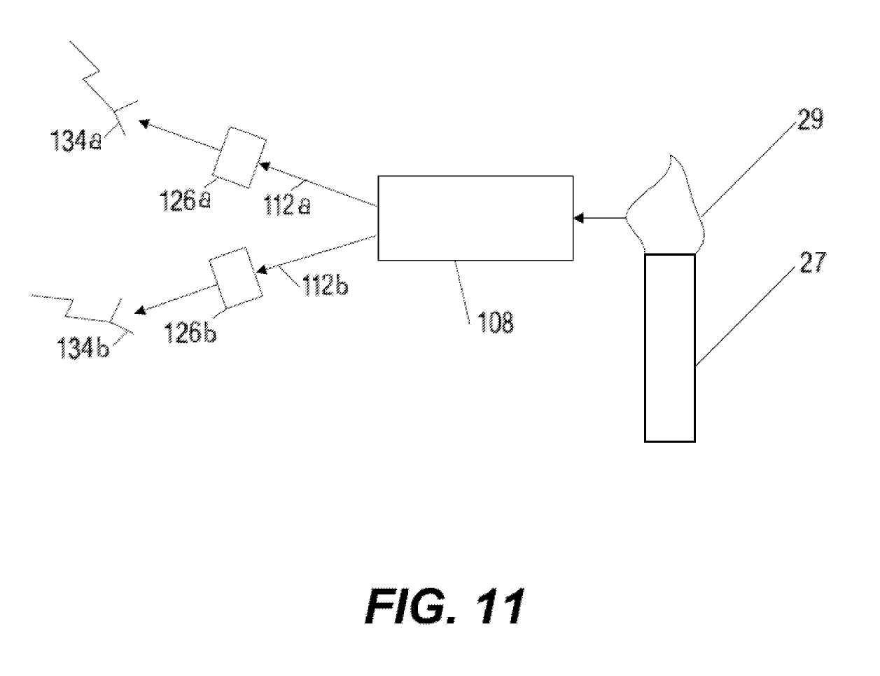

Referring now to FIG. 11, which provides a nonlimiting example of a system of the present invention, a plurality of MOEs may be utilized at the same time, by illuminating them all with the telescope 108. In other words, different portions 112a, 112b of beam 112 may illuminate different MOEs 126a, 126b and provide information to different detectors 134a, 134b. In this manner, any number of MOEs may be used in parallel.

In some embodiments, systems of the present invention may include an optical calculation device for performing a regression calculation on the interacted light, the device being responsive to interacted light (as described herein to include flames and light passing through with fluids relating to oilfield operations) incident thereon to produce at least one output light signal; and a signal processing arrangement for determining the at least one property of the fluid from the at least one output light signal. In some embodiments, the calculation device of the systems of the present invention may include a multivariate optical element that comprises a plurality of optical refraction layers, the layers manifesting a multivariate calculation wherein the result of the calculation correlates with a property of the fluid. In some embodiments, systems of the present invention may further include a housing containing at least the device, a turbulence generator in a pipe system upstream of the area in which the device is monitoring (e.g., in a pipe or flare stack), a plurality of said optical calculation devices (for detecting the same or a variety of properties), an internal calibration arrangement for correcting for drift in value of the calculation value, a processing arrangement for determining the at least one property according to a corrected drift value, or any combination thereof.

In some embodiments, systems of the present invention may include a connection to the oilfield operation that operably is capable of changing at least one aspect of the oilfield operation. Said aspects may be a hardware aspects (e.g., valves, pumps, motors, and the like) and/or operational aspects (e.g., flow rates, flow directions, drilling speeds, drilling pressures, fluid pressures, temperatures, components of a fluid stream, and the like).

In some embodiments, systems of the present invention may include an optical calculation device arranged for generating first and second signals representing respective transmitted and reflected light from the device and a signal processing arrangement adapted to process the first and second signals to determine at least one property of the fluid.

In some embodiments, the fluid relating to an oilfield operation being monitored may include an indicator in the fluid for indicating a parameter of the fluid.

Some embodiments of the present invention may include receiving interacted light (light having interacted with a fluid relating to an oilfield operation) to an optical calculation device, performing a regression calculation on the interacted light to produce at least one output light signal, and determining the at least one property of the fluid from the at least one output light signal. Some embodiments of the present invention may further include changing at least one aspect of the oilfield operation based on the at least one property.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

The invention claimed is:

1. A method comprising:
directing interacted light that comprises light having passed through a fluid relating to an oilfield operation to an iris;
performing a regression calculation on the interacted light with an optical calculation device responsive to the interacted light incident thereon to produce at least one output light signal; and
determining the at least one property of the fluid from the at least one output light signal.

2. The method of claim 1 further comprising:
changing at least one aspect of the oilfield operation based on the at least one property.

3. The method of claim 1, wherein the step of performing the regression calculation includes generating first and second signals representing respective transmitted and reflected light from the optical calculation device and wherein said determining step includes processing the first and second signals.

4. The method of claim 3, wherein the step of performing the regression analysis comprises applying the interacted light from the iris to a plurality of stacked optical layers.

5. The method of claim 4, wherein the at least one property is selected from the group consisting essentially of asphaltene, saturates, resins, aromatics, solid particulate content, hydrocarbon composition and content, gas composition C₁-C₆ and content, CO₂, H₂S and correlated, a gas component of a gas phase of petroleum, total stream percentage of water, gas, oil, solid particles, solid types, oil finger printing, reservoir continuity, oil type, and water elements, and wherein the water elements consist essentially of ion composition and content, anions, cations, salinity, organics, pH, mixing ratios, tracer components, and contamination.

6. The method of claim 1 further comprising:
directing the interacted light through a telescope prior to directing the interacted light through the iris.

7. The method of claim 6 further comprising:
generating spectroscopic data from the interacted light with a conventional spectroscopic instrument and then using that generated spectroscopic data for producing said optical calculation device.

8. The method of claim 6 further comprising:
providing turbulence to the fluid upstream of said flame to provide homogeneous distribution of different components in the fluid fueling said flame.

9. The method of claim 1, wherein the performing step includes applying the interacted light to a plurality of stacked optical layers forming a multivariate optical calculation device.

10. The method of claim 9 further comprising:
providing a plurality of said multivariate optical calculation devices and applying the interacted light to each said device, each device corresponding to a different property to be determined.

11. The method of claim 1 further comprising:
providing an internal calibration to correct for drift in value of the calculation value and then determining the at least one property according to that corrected drift value.

12. A method comprising:
directing interacted light that comprises light from a flame that comprises a fluid relating to an oilfield operation to an iris;
performing a regression calculation on the interacted light with an optical calculation device responsive to the interacted light incident thereon to produce at least one output light signal; and
determining the at least one property from the at least one output light signal.

13. A system comprising:
interacted light comprising at least one selected from the group consisting of light having interacted with fluid relating to an oilfield operation, light emitted from a flame comprising fluid relating to an oilfield operation, and any combination thereof;
an optical calculation device for performing a regression calculation on the interacted light, the device being responsive to the interacted light incident thereon to produce at least one output light signal; and
a signal processing arrangement for determining at least one property of the fluid from the at least one output light signal.

14. The system of claim 13, wherein the calculation device includes a multivariate optical element that comprises a plurality of optical refraction layers, the layers manifesting a multivariate calculation wherein the result of the calculation correlates with a property of the fluid.

15. The system of claim 13 further comprising:
providing an indicator in the fluid for indicating a parameter of the fluid.

16. The system of claim 13 further comprising:
a housing containing at least the device.

17. The system of claim 13 further comprising:
a turbulence generator in a pipe system upstream from the flame.

18. The system of claim 13 further comprising:
at least one turbulence generator in a pipe system for mixing the fluid upstream relative to the device.

19. The system of claim 13, wherein the device is arranged for generating first and second signals representing respective transmitted and reflected light from the device; and wherein the signal processing arrangement is adapted to process the first and second signals to determine the at least one property.

20. The system of claim 13 further comprising:
a plurality of said optical calculation devices, wherein the interacted light is applied to each said device, each device corresponding to a different property to be determined.

21. The system of claim 13 further comprising:
an internal calibration arrangement for correcting for drift in value of the calculation value, wherein the processing arrangement determines the at least one property according to that corrected drift value.

22. The system of claim 13 further comprising:
a connection to the oilfield operation that operably is capable of changing at least one aspect of the oilfield operation.
